(19) United States
(12) Patent Application Publication
(10) Pub. No.: US 2026/0132064 A1
(43) Pub. Date: May 14, 2026

(54) A METHOD FOR IMPROVING BIOLOGICAL MANGANESE OXIDATION PERFORMANCE OF MANGANESE-OXIDIZING BACTERIA AND APPLICATION THEREOF

(71) Applicant: Qingdao University of Technology, Qingdao (CN)

(72) Inventors: HUAWEI WANG, Qingdao (CN); XIQING CUI, Qingdao (CN); ZHIBO YANG, Qingdao (CN); YANAN WANG, Qingdao (CN); YINGJIE SUN, Qingdao (CN); YUJUN WANG, Qingdao (CN); SHUPENG LI, Qingdao (CN); YING GAO, Qingdao (CN)

(21) Appl. No.: 19/031,961

(22) Filed: Jan. 18, 2025

(30) Foreign Application Priority Data
Nov. 13, 2024 (CN) 202411614470.7

(51) Int. Cl. C02F 3/34; C01B 32/05; C02F 1/28; C02F 101/20; C12N 1/38

(52) U.S. Cl. CPC C02F 3/348; C01B 32/05; C02F 1/281; C12N 1/38; C02F 2101/20; C02F 2305/06

(57) ABSTRACT

The invention, as a sewage treatment technique, provides a method for improving biological manganese oxidation performance of manganese-oxidizing bacteria and application thereof. By adding biochar, it improves the biological manganese oxidation performance of the manganese-oxidizing bacteria. It can increase the oxidation rate of the divalent manganese after adding the biochar first and the divalent manganese next into the manganese-oxidizing bacteria. The heavy metals, accordingly, in the wastewater could be removed efficiently. The biochar, as an electron shuttle, can prompt the electron transfer and accelerate the oxidation rate of divalent manganese, as well as the gene abundance and expression ability of manganese-oxidizing bacteria and the extracellular superoxide concentration. The biological activity of the manganese-oxidizing bacteria and the oxidation of divalent manganese are enhanced. In addition, the biochar is abundant and low-cost, which may reutilize resources and reduce the treatment cost. It is easy to prepare, release and use it in practice.

### CROSS-REFERENCE TO RELATED APPLICATION

This application claims priority of Chinese Patent Application No. 202411614470.7, filed on Nov. 13, 2024, the entire contents of which are incorporated herein by reference.

### TECHNICAL FIELD

The invention relates to the technical field of sewage treatment, in particular to a method for improving the biological manganese oxidation performance of manganese-oxidizing bacteria and application thereof.

### BACKGROUND

Manganese-oxidizing bacteria (MnOB) are a group of microorganisms that can rapidly catalyze the oxidation of Mn (II) and induce the formation of biological manganese oxides (BMO). Manganese-oxidizing bacteria can induce the production of biological manganese oxides by biosynthesis under neutral, normal temperature and atmospheric conditions. Compared with chemically synthesized manganese oxides, biosynthesized BMO has smaller particle size, weaker crystallization, higher Mn valence, more holes in the octahedral structure, larger specific surface area and stronger reactivity. In the natural environment, the biological oxidation rate of divalent manganese by manganese-oxidizing bacteria is 10⁵ times higher than that of chemical oxidation. In addition, due to their special crystal structures and surface chemical properties, biological manganese oxides have strong adsorption capacity for a variety of heavy metal ions, and can also adsorb and degrade some organic pollutants, such as pesticides, endocrine disruptors, antibiotics, polycyclic aromatic hydrocarbons and so on. Therefore, manganese-oxidizing bacteria play an important role in the remediation of polluted environment.

In the process of inducing biological manganese oxides by manganese-oxidizing bacteria, the biological manganese oxidation performance plays a vital role, which can be reflected by the oxidation rate of divalent manganese and the formation rate of biological manganese oxides. Many environmental factors can affect the performance of biological manganese oxidation, such as DO, pH, Eh, temperature, concentration of divalent manganese, strain type and so on. Overall, the faster the biological manganese oxidation rate is, the higher the biological manganese oxide is, and the more conducive to the remediation of pollutants.

At present, there are mainly two ways to improve the performance of biological manganese oxides. Firstly, the stimulation factor is added to promote the manganese oxidation reaction and accelerate the oxidation rate of divalent manganese and the formation rate of manganese oxides. Secondly, the addition of the carrier increases the contact area and improves the physical structure of the reaction system, thereby improving the manganese oxidation performance. However, the first method to improve the performance of biological manganese oxidation by external stimulation factors is difficult to achieve in the actual remediation project, and the improvement is very limited, so the second method is mostly used. Therefore, it is urgent to develop a green, low-cost and no secondary pollution method to improve the performance of biological manganese oxidation.

### SUMMARY

The invention aims to provide a method for improving the biological manganese oxidation performance of manganese-oxidizing bacteria and application thereof, which can obviously increase the oxidation rate of divalent manganese and improve the removal efficiency of heavy metals in wastewater.

In order to achieve the purpose of the invention, the invention provides the following technical scheme:

The invention provides a method for improving the biological manganese oxidation performance of manganese-oxidizing bacteria, which comprises the following steps:
(1) Performing high-temperature treatment to the pretreated straws under the atmosphere of CO₂, and cooling, grinding and sieving to obtain black powder. Then performing acid treatment to the black powder, and washing and drying to obtain biochar powder;
(2) Inoculating manganese-oxidizing bacteria in a manganese-oxidizing bacteria culture medium, then adding the biochar in the step (1) into the manganese-oxidizing bacteria culture medium for culture, and adding a divalent manganese solution after the culture is finished.

Preferably, in step (1), the straw pretreatment method comprises:

Drying the straws at 60-80° C. to reduce the moisture content of the straws to 10-15%, and crushing the straws until the length of the straws is 1-5 cm.

Preferably, in step (1), the temperature of the high-temperature treatment is 300 to 600° C., the treatment time is 7 to 12 hours, and the mesh number of the sieving is 60 to 300 meshes.

Preferably, in step (1), the step of acid treatment of the black powder comprises:

Adding the black powder into a hydrochloric acid solution with the concentration of 0.2-1.0 mol/L, and stirring for 8-24 hours, wherein the mass volume ratio of the black powder to the hydrochloric acid solution is 1 g: 10-20 mL.

Preferably, in step (2), the manganese-oxidizing bacteria is P. putida strain MnB1; the concentration of the manganese-oxidizing bacteria for inoculation is 1˜3×10⁷ CFU/ml, the inoculation amount of the manganese-oxidizing bacteria is 2-10%, and the PH of the manganese-oxidizing bacteria culture medium is 6.0-7.5; the addition amount of the biochar is 1.0-10.0 g/L, and the culture time is 1-2 days; the concentration of divalent manganese is 2.5-12.0 mg/L.

The invention also provides the application of the method for improving the biological manganese oxidation performance of manganese-oxidizing bacteria in sewage purification, and the application method comprises:

Inoculating the manganese-oxidizing bacteria in the sewage, adding the biochar for culture, and adding the divalent manganese solution.

Preferably, the PH of the sewage is 6.0-7.5; the inoculation amount of the manganese-oxidizing bacteria in the sewage is 2-10%, and the concentration of the manganese-oxidizing bacteria is 1˜3×10⁷ CFU/ml; the addition amount of the biochar is 1.0 to 10.0 g/L, and the culture time is 1 to 2 days; the concentration of the divalent manganese is 5 to 12.0 mg/L.

Compared with the prior art, the invention has the following beneficial effects:
(1) The invention, by adding the biochar, improves the biological manganese oxidation performance of the manganese-oxidizing bacteria. It can increase the oxidation rate of the divalent manganese after adding the biochar first and the divalent manganese next into the manganese-oxidizing bacteria. In this case, the heavy metals in the wastewater could be removed more efficiently. The biochar can be used as an electron shuttle to improve the electron transfer ability and accelerate the oxidation rate of divalent manganese. The biochar can also enhance the gene abundance and expression ability of manganese-oxidizing bacteria and the extracellular superoxide concentration. It can improve the biological activity of the manganese-oxidizing bacteria, and accelerate the oxidation of divalent manganese.
(2) As a green environmental protection material, biochar will not cause secondary pollution to soil, water and air in the use process. In addition, the biochar has wide sources and low cost. It can realize the reutilization of resources and reduce the treatment cost. It is easy to prepare and put in, and is convenient to use in practical application.
(3) Compared with free bacteria, the addition of biochar provides a biological carrier for manganese-oxidizing bacteria, which is conducive to the attachment and growth of manganese-oxidizing bacteria, thus creating a good living environment for manganese-oxidizing bacteria.

### BRIEF DESCRIPTION OF DRAWINGS

To more clearly illustrate the embodiments of the present invention or the technical solutions in the prior art, the following will briefly introduce the drawings used in the embodiments. Obviously, the drawings in the following description are only some embodiments of the present invention. For those of ordinary skill in the art, other FIGs may also be derived from these FIGs.

FIG. 1 is a scanning electron micrograph of the biochar according to Embodiment 1 of the present invention;

FIG. 2 is a scanning electron micrograph of the biochar after colony attachment in Embodiment 1 of the present invention.

### DETAILED DESCRIPTION

Various exemplary embodiments of the present invention will now be described in detail. This detailed description should not be taken to be limiting of the invention, but rather should be taken as a more detailed description of certain aspects, features, and embodiments of the present invention.

The terminology used herein is for the purpose of describing particular embodiments only and is not intended to be limiting of the invention. In addition, with respect to numerical ranges in the present invention, each intermediate value between the upper and lower limits of the range is also specifically disclosed. An intermediate value within any stated value or range and every lesser range between any other stated value or intermediate value within a stated range is also encompassed within the invention. The upper and lower limits of these lesser ranges may be independently included or excluded from the range.

Unless otherwise indicated, all technical and scientific terms used herein have the same meaning as commonly understood by one of ordinary skill in the art to which this invention belongs. While only the preferred methods and materials have been described herein, any methods and materials similar or equivalent to those described herein can be used in the practice or testing of the present invention. All documents mentioned in this specification are incorporated by reference for the purpose of disclosing and describing the methods and/or materials associated with said documents. In the event of a conflict with any incorporated documents, the contents of this specification shall prevail.

It will be apparent to those skilled in the art that various modifications and variations can be made in the specific embodiments described herein without departing from the scope or spirit of the invention. Other embodiments will be apparent to those skilled in the art from this description of the invention. The description and embodiments of the present invention are exemplary only.

As used herein, the terms “comprising”, “including”, “having”, “containing” and the like are open-ended terms that mean including, but not limited to.

The invention relates to a method for improving the biological manganese oxidation performance of manganese-oxidizing bacteria, which comprises the following steps:
(1) Performing high-temperature treatment to the pretreated straws under the atmosphere of CO₂, and cooling, grinding and sieving to obtain black powder. Then performing acid treatment to the black powder, and washing and drying to obtain biochar powder;
(2) Inoculating manganese-oxidizing bacteria in a manganese-oxidizing bacteria culture medium, then adding the biochar in the step (1) into the manganese-oxidizing bacteria culture medium for culture, and adding a divalent manganese solution after the culture is finished.

### Embodiment 1

Embodiment 1 of the present invention provides a method for improving the biological manganese oxidation performance of manganese-oxidizing bacteria, which comprises the following steps:

(1) Preparation of P. putida Strain MnB1 Bacterial Fluid:

Inoculating the P. putida strain MnB1 into the P. putida enrichment medium according to the transfer amount of 3% by volume, and then carrying out aerobic enrichment culture for 2 days under horizontal oscillation (150 rpm) at the temperature of 30° C. to obtain the P. putida strain MnB1 bacterial fluid. The bacterial concentration is 1×10⁷ CFU/ml.

The medium composition is as follows: yeast extract, 0.5 g/L; acid hydrolyzed casein, 0.5 g/L; glucose, 1 g/L; calcium chloride dihydrat, 0.29 g/L; magnesium sulfate heptahydrate, 0.82 g/L; ferric chloride, 0.001 g/L; trace elements, 1 mL; pH=7.0. The trace elements are as follows: copper sulfate pentahydrate, 2.49 g/L; zinc sulfate heptahydrate, 12.65 g/L; cobalt chloride hexahydrat, 4.76 g/L; sodium molybdate dihydrate, 3.15 g/L.

The selected P. putida strain MnB1 is purchased from the American Type Culture Collection (ATCC) as 23483.

### (2) Preparation of Biochar:

Drying the straws at 80° C. to reduce the moisture content of the straws to 15%, and crushing the straws until the length of the straws is 5 cm.

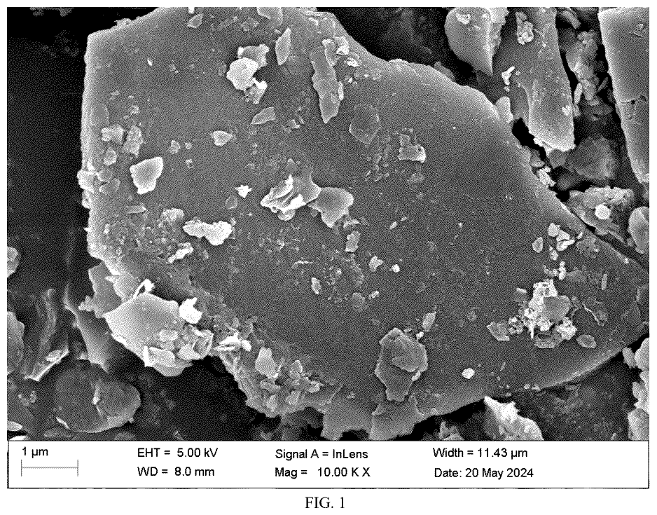

Putting 100 g of crushed straw into a pyrolysis furnace, introducing CO₂ protective gas, heating to 600° C., continuously keeping the temperature for 10 hours, cooling, grinding, and sieving with a 300-mesh sieve to obtain black powder; adding the obtained black powder into 1.0 mol/L hydrochloric acid solution at a mass/volume ratio of 1 g:10 mL, stirring for 24 hours, filtering, washing with 300 ml deionized water to pH 8.0, and lyophilizing in a freeze dryer for 24 hours to obtain biochar powder. The scanning electron microscope image is shown in FIG. 1.

As FIG. 1 shows, the addition of biochar provides a larger specific surface area for manganese-oxidizing bacteria, which is conducive to the attachment of microorganisms and provides a good environment for the growth of microorganisms.

### (3) Improving the Biological Manganese Oxidation Performance of Manganese-Oxidizing Bacteria:

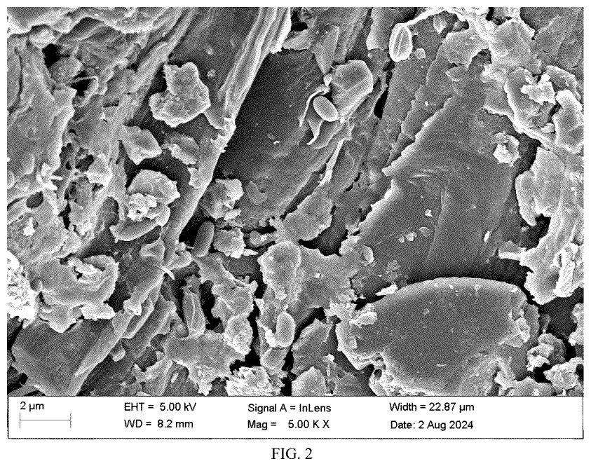

Sterilizing and cooling 250 ml of ATCC279 #culture medium system, adding HEPES buffer, adjusting the pH to 7.2, adding the manganese-oxidizing bacteria cultured in step 1) according to 3% of the inoculation ratio, adding 5.0 g/L of biochar prepared in step 2), culturing for 2 days (after adding the biochar, the colony adheres to its surface as shown in FIG. 2), adding divalent manganese to a final concentration of 12 mg/L, and culturing the mixture in a vertical full temperature shaking culture machine at 30° C. and at 120 rpm.

### Embodiment 2

The method of step (3) in Embodiment 1 is applied in Embodiment 2 of the present invention to determine the BMO formation rate and the manganese oxidation kinetic rate constant under varying doses of biochar. The difference between Embodiment 2 and Embodiment 1 is that the biochar dosages in Embodiment 2 are 0 g/L, 1.0 g/L, 2.5 g/L, 5.0 g/L and 10.0 g/L, respectively. The samples are taken at 4 h, 8 h, 12 h, 24 h, 36 h and 48 h respectively, the concentrations of divalent manganese and BMO are determined, and the oxidation kinetic rate constant of divalent manganese and the formation rate of BMO are calculated. The results are shown in Table 1.

TABLE 1
Manganese Oxidation Kinetic Rate Constants and BMO Formation
Rate Constants at Different Biochar Dosages

| | Biochar | Divalent manganese | BMO formation |
|---|---|---|---|
| | content | oxidation rate constant | rate constant |
| | (g/L) | (mg · L⁻¹ · h⁻¹) | (mg (MnO2) · L⁻¹ · h⁻¹) |
| Control | 0.0 | 0.11 | 0.41 |
| group | | | |
| Experimental | 1.0 | 0.17 | 0.76 |
| group | 2.5 | 0.20 | 0.95 |
| | 5.0 | 0.23 | 1.25 |
| | 10.0 | 0.21 | 1.14 |

As Table 1 shows, the divalent manganese oxidation rate becomes higher when biochar is instilled, so does BMO formation rate, compared with the control group. When the biochar dosage is 5.0 g/L, the divalent manganese oxidation rate constant is 0.23 mg·L⁻¹·h⁻¹, and the BMO formation rate is 1.25 mg (MnO₂)·L⁻¹·h⁻¹, which are 110% and 205% higher than those of the control group, respectively.

### Embodiment 3

The method of step (3) in Embodiment 1 is applied in Embodiment 3 of the present invention to determine the rate of BMO formation and the manganese oxidation kinetic rate constant under various divalent manganese additions and concentrations. Different from Embodiment 1, the final concentrations of divalent manganese in Embodiment 3 are 2.5 g/L, 5.0 g/L, 10.0 g/L and 12.0 g/L, respectively, and the results are shown in Table 2.

TABLE 2
Manganese Oxidation Kinetic Rate Constants and
BMO Generation Rate Constants under Different
Concentration and Addition of Divalent Manganese

| Mn²⁺ | Divalent manganese | BMO formation |
|---|---|---|
| concentration | oxidation rate constant | rate constant |
| (mg/L) | (mg · L⁻¹ · h⁻¹) | (mg (MnO2) · L⁻¹ · h⁻¹) |
| 2.5 | 0.04 | 0.22 |
| 5.0 | 0.08 | 0.44 |
| 10.0 | 0.24 | 0.80 |
| 12.0 | 0.47 | 1.24 |

As Table 2 shows, the concentration of Mn²⁺ increases, so do the divalent manganese oxidation rate constant and the BMO formation rate. When the final concentration of Mn²⁺ is 12.0 mg/L, the divalent manganese oxidation rate constant is 0.47 mg·L⁻¹·h⁻¹, and the BMO formation rate is 1.24 mg (MnO₂)·L⁻¹·h⁻¹. Compared with the final Mn²⁺ concentration of 2.5 mg/L, the divalent manganese oxidation rate constant is increased by 11 times, and the BMO formation rate is increased by 5 times.

### Embodiment 4

The method of step (3) in Embodiment 1 is applied in Embodiment 4 of the present invention to determine the BMO formation rate constant and the manganese oxidation kinetic rate constant under various PH settings. Different from Embodiment 1, the PH in Embodiment 4 is 6.0, 7.2 and 7.6 respectively, and the results are shown in Table 3.

TABLE 3
Manganese Oxidation Kinetic Rate Constants and BMO Formation
Rate Constants under Different pH Conditions

| | Divalent manganese | BMO formation |
|---|---|---|
| | oxidation rate constant | rate constant |
| pH | (mg · L⁻¹ · h⁻¹) | (mg (MnO2) · L⁻¹ · h⁻¹) |
| 6.0 | 0.41 | 0.61 |
| 7.2 | 0.50 | 0.87 |
| 7.6 | 0.38 | 0.00 |

As Table 3 shows, within a certain range, as the pH increases, the divalent manganese oxidation rate constant also increases, so does the BMO formation rate. The divalent manganese oxidation rate constant and the BMO formation rate reach the maximum at pH 7.2. The divalent manganese oxidation rate constant is 0.50 mg·L⁻¹·h⁻¹, and the BMO formation rate is 0.87 mg (MnO₂)·L⁻¹·h⁻¹.

### Embodiment 5

The method of Embodiment 1 is applied in Embodiment 5 of the present invention, to determine the Cd removal efficiency of the Cd-containing wastewater, and the specific method is as follows:

Taking 1 L of Cd-containing wastewater with the initial concentration of 1.0 mg/L, adjusting the pH of the wastewater to 7.2, adding the manganese-oxidizing bacteria strain P. putida strain MnB1 according to the inoculation ratio of 3%, adding the biochar according to the dosage of 5.0 g/L, and culturing for 2 days; adding divalent manganese to a final concentration of 12 mg/L, then placing in an incubator for 48 hours at 30° C. and at 140 rpm, measuring the Cd concentration periodically at different time periods, and calculating the removal efficiency of Cd. The results are shown in Table 4.

TABLE 4
Cd Removal Rate

| Time (h) | 12 | 24 | 36 | 48 |
|---|---|---|---|---|
| Cd removal rate | 74.52 | 86.34 | 92.46 | 96.75 |
| (%) | | | | |

As Table 4 shows, with the change of time, the removal rate of Cd gradually increases, and the removal effect is better. In the first 12 hours, the removal rate has reached 74.52%, indicating that the removal of Cd has made rapid progress during this period. In 24-48 hours, the removal rate continues to increase, but the growth rate slows down. The maximum removal rate is 96.75% at 48 h, which is 22.23% higher than that at 12 h.

### Embodiment 6

The method of Embodiment 5 is applied in Embodiment 6 of the present invention, to determine the Cd removal efficiency of the Cd-containing wastewater. Unlike Embodiment 5, the concentration of the Cd-containing wastewater in Embodiment 6 is 2.0 mg/L, and the results are shown in Table 5.

TABLE 5
Cd Removal Rate

| Time (h) | 12 | 24 | 36 | 48 |
|---|---|---|---|---|
| Cd removal rate | 70.36 | 82.57 | 90.40 | 92.58 |
| (%) | | | | |

As Table 5 shows, with the change of time, the removal rate of Cd gradually increases, and the removal effect is better. In the first 12 hours, the removal rate has reached 70.36%, indicating that the removal of Cd has made rapid progress during this period. In 24-48 hours, the removal rate continues to increase, but the growth rate slows down. The maximum removal rate is 92.58% at 48 h, which is 22.22% higher than that at 12 h.

### Embodiment 7

The method of Embodiment 5 is applied in Embodiment 7 of the present invention to determine the Cd removal efficiency of the Cd-containing wastewater. Unlike Embodiment 5, the concentration of the Cd-containing wastewater in Embodiment 7 is 4.0 mg/L, and the results are shown in Table 6.

TABLE 6
Cd Removal Rate

| Time (h) | 12 | 24 | 36 | 48 |
|---|---|---|---|---|
| Cd removal rate | 70.39 | 80.97 | 88.32 | 93.76 |
| (%) | | | | |

As Table 6 shows, with the change of time, the removal rate of Cd gradually increases, and the removal effect is better. In the first 12 hours, the removal rate has reached 70.39%, indicating that the removal of Cd has made rapid progress during this period. In 24-48 hours, the removal rate continues to increase, but the growth rate slows down. The maximum removal rate is 93.76% at 48 h, which is 23.37% higher than that at 12 h.

The above is only a preferred embodiment of the present invention. Please note that those skilled in the art can make several improvements and modifications without departing from the principles of the present invention, and these improvements and modifications should also be regarded as the scope of protection of the present invention.

1. A method for improving the biological manganese oxidation performance of manganese-oxidizing bacteria, which is characterized in that it comprises the following steps:
(1) Performing high-temperature treatment to the pretreated straws under the atmosphere of CO₂, and cooling, grinding and sieving to obtain black powder. Then, performing acid treatment to the black powder, and washing and drying to obtain biochar powder;
(2) Inoculating manganese-oxidizing bacteria in a manganese-oxidizing bacteria culture medium, then adding the biochar in step (1) into the manganese-oxidizing bacteria culture medium for culture, and adding a divalent manganese solution after the culture is finished.

2. In accordance with the “method for improving the biological manganese oxidation performance of manganese-oxidizing bacteria” as described in claim 1, it is characterized in that in step (1), the straw pretreatment method comprises:
Drying the straws at 60-80° C. to reduce the moisture content of the straws to 10-15%, and crushing the straws until the length of the straws is 1-5 cm.

3. In accordance with the “method for improving the biological manganese oxidation performance of manganese-oxidizing bacteria” as described in claim 1, it is characterized in that in step (1), the temperature of the high-temperature treatment is 300 to 600° C., the treatment time is 7 to 12 hours, and the mesh number of the sieving is 60 to 300 meshes.

4. In accordance with the “method for improving the biological manganese oxidation performance of manganese-oxidizing bacteria” as described in claim 1, it is characterized in that in step (1), the step of acid treatment of the black powder comprises:
Adding the black powder into a hydrochloric acid solution with the concentration of 0.2-1.0 mol/L, and stirring for 8-24 hours, wherein the mass volume ratio of the black powder to the hydrochloric acid solution is 1 g: 10-20 mL.

5. In accordance with the “method for improving the biological manganese oxidation performance of manganese-oxidizing bacteria” as described in claim 1, it is characterized in that in step (2), the manganese-oxidizing bacteria is P. putida strain MnB1; the concentration of the manganese-oxidizing bacteria for inoculation is 1˜3×10⁷ CFU/ml, the inoculation amount of the manganese-oxidizing bacteria is 2-10%, and the PH of the manganese-oxidizing bacteria culture medium is 6.0-7.5; the addition amount of the biochar is 1.0-10.0 g/L, and the culture time is 1-2 days; the concentration of divalent manganese is 2.5-12.0 mg/L.

6. An application of the “method for improving the biological manganese oxidation performance of manganese-oxidizing bacteria” in sewage purification, which is characterized in that the application method comprises:
Inoculating the manganese-oxidizing bacteria into the sewage, adding the biochar for culture, and then adding the divalent manganese solution.

7. In accordance with the application of the “method for improving the biological manganese oxidation performance of manganese-oxidizing bacteria in sewage purification” as described in claim 6, it is characterized in that the PH of the sewage is 6.0-7.5; the inoculation amount of the manganese-oxidizing bacteria in the sewage is 2-10%, and the concentration of the manganese-oxidizing bacteria is 1˜3×10⁷ CFU/ml; the addition amount of the biochar is 1.0 to 10.0 g/L, and the culture time is 1 to 2 days; the concentration of the divalent manganese is 5 to 12.0 mg/L.
